(19) United States
(12) Patent Application Publication
(10) Pub. No.: US 2004/0122430 A1
(43) Pub. Date: Jun. 24, 2004

(54) Implant

(76) Inventor: Henrik Hansson, Vreta Kloster (SE)

Correspondence Address: Tarolli Sundheim Covell & Tummino, Suite 1111, 526 Superior Avenue, Cleveland, OH, 44114-1400, US

(21) Appl. No.: 10/475,572

(22) PCT Filed: Apr. 25, 2002
(86) PCT No.: PCT/SE02/00806

(30) Foreign Application Priority Data
May 8, 2001 (SE) 0101585

(51) Int. Cl. A61B 17/56

(52) U.S. Cl. 606/69

(57) ABSTRACT

The present invention relates to an implant for fixation of bone fragments at femur fractures, preferably subtrochantary femur fractures, said implant comprising an elongated member (9) and integral therewith, a sleeve like member. The elongated member (9) has an inner side (14) and an outer side (15), said inner side 814) being adapted to engage the bone fragment (3) including the femoral shaft (1). The elongated member (9) of the implant (8) has elongated holes (16) for screws (11) having heads (18) with contact surfaces (19) which are adapted to engage contact surfaces (17) on the elongated member (9). Each contact surface (17) on the elongated member (9) is inclined relative to the inner side (14) of said elongated member (9) such that the distance (A) between said contact surface (17) at the rear portions (16a) of the elongated hole (16) and said inner side (14) is greater than the distance (B) between said contact surface (17) and said inner side (14) at front portions (16b) of the elongated hole (16), whereby each screw (11) can move in forward direction (F) in said elongated hole (16) without said movement being counteracted or substantially counteracted by the contact surface (17) of the elongated member (9).

The present invention relates to an implant for fixation of bone fragments at femur fractures, preferably subtrochantary femur fractures. The implant comprises an elongated member and integral therewith, a sleeve like member. The sleeve like member is adapted for location at a bone fragment including the head of femur and wherein the elongated member is adapted for location at a bone fragment including the femoral shaft. The implant comprises a bone screw having a threaded portion which is adapted to be screwed into and secured to the femoral head. The sleeve like member has a hole which is adapted for receiving a portion of the bone screw. The elongated member has an inner side and an outer side, said inner side being adapted to engage the bone fragment including the femoral shaft. The elongated member of the implant has elongated holes for screws by means of which said elongated member can be tightened to the bone fragment with the femoral shaft. The screws have heads with contact surfaces which are adapted to engage contact surfaces on the elongated member. The elongated holes are provided such that the screws can be moved therein in a forward direction towards the sleeve like member of the implant during displacement of the bone fragment with the femoral shaft in said forward direction relative to the elongated member. The screws are adapted for initial location in such rear portions of the elongated holes which are situated farthest away from the sleeve like member. The screws are adapted for tightening when they are located in said rear portions of the elongated holes such that the contact surfaces on their heads engage the contact surfaces of the elongated member for fixation of said elongated member at the bone fragment with the femoral shaft.

The publication U.S. Pat. No. 2,761,444 refers to an implant of the abovementioned type. Such implants have elongated holes in the so called plate for screws through which the plate is attached to the femur shaft. Due to instability in the fracture, bone absorption and eventual other reasons, the femur shaft should be able to move in upwards direction relative to the plate of the implant. This movement of the femur shaft in upwards direction relative to the plate is possible while the screws can move in the elongated holes of the plate.

Initially, it is important that the screws are tightened with such a great tightening force that the plate is stably fixed at the femoral shaft. This stable fixation of the plate at the femoral shaft means however, that the contact surfaces of the plate, engaged by the screws, later counteract a movement of the screws and thereby of the femoral shaft relative to the plate. The more stable the plate has been initially fixed at the femoral shaft, the more difficult is it later for the femoral shaft to move relative to the plate. This contrasting relationship is yet to be solved.

The object of the present invention has been to eliminate the abovementioned problem and this has been done by providing the implant primarily with the characterizing features of subsequent claim 1.

Since the implant, has been provided with said characterizing features, it is achieved that the screws of the implant can be tightened with sufficient great tightening force for initial stable fixation of the plate at the femoral shaft without the contact surfaces of the plate for the screws, through this tightening of the screws, later prevent or counteract the movements of the screws in the elongated holes and thereby, the movements of the femoral shaft relative to the plate.

The invention will be further described below with reference to the accompanying drawings, in which

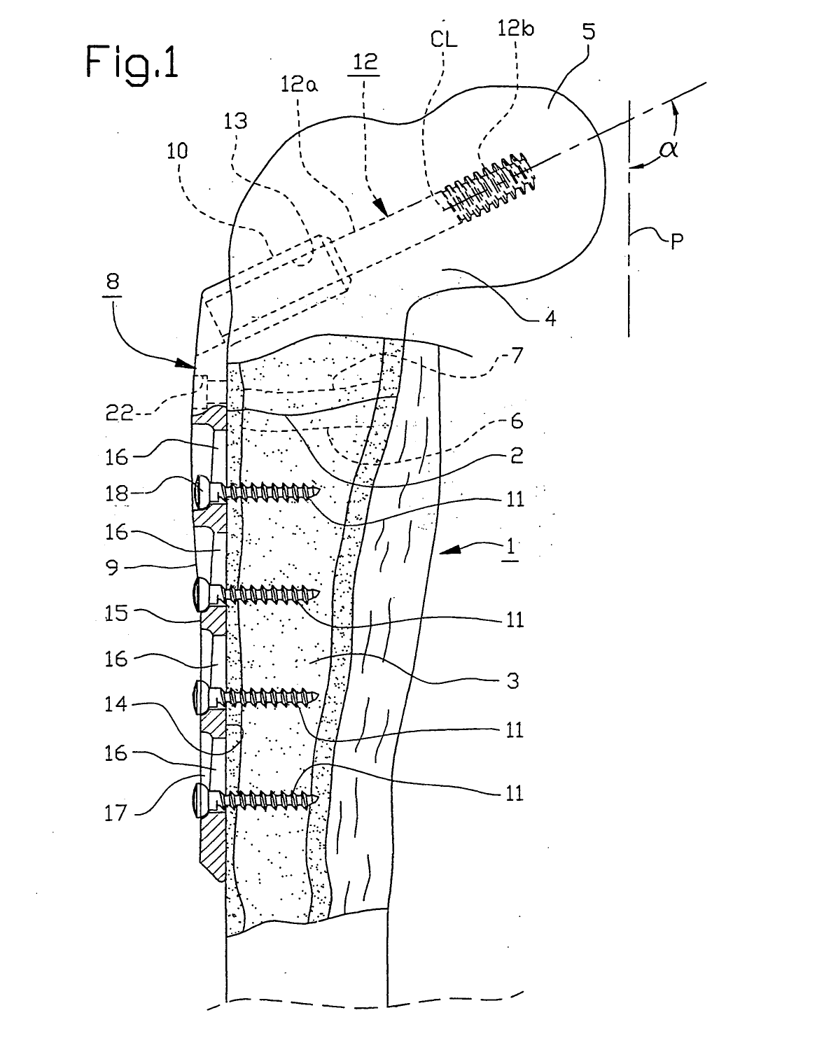

FIG. 1 is a partly longitudinal section of an implant according to the invention;

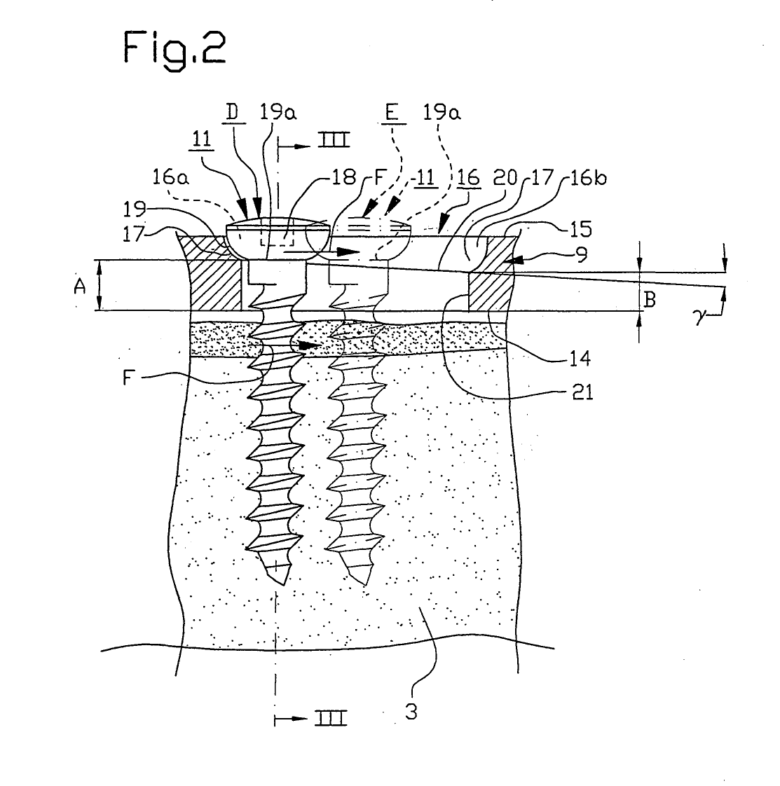

FIG. 2 is an enlarged section of a part of the implant of FIG. 1; and

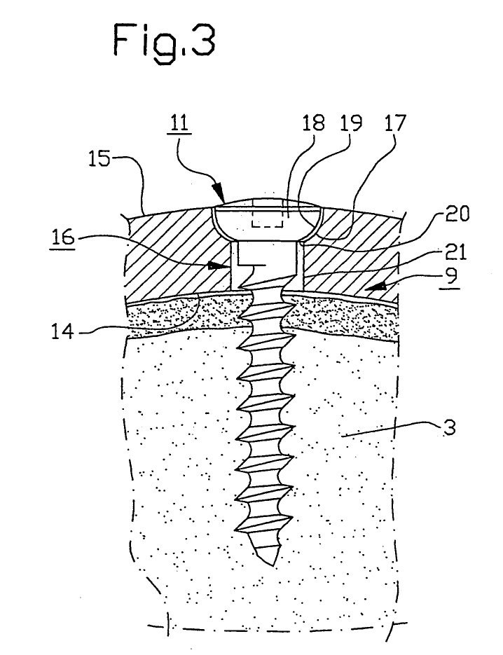

FIG. 3 is a section III-III of the part of the implant illustrated in FIG. 2.

In FIG. 1, a thighbone (femur) is schematically illustrated, the femoral shaft 1 of which has a fracture 2, more specifically in this case a subtrochantary fracture. The bone fragment 3 beneath the femur fracture 2 include essential parts of the femoral shaft 1, while the bone fragment 4 above said femur fracture 2 includes the femoral head 5. On both sides of the femur fracture 2 and in direct connection thereto, there are injured bone fragment portions 6 and 7.

In order to fix the bone fragments 3, 4 relative to each other, an implant 8 is used, which is secured in the bone fragment 3 as well as the bone fragment 4. The implant 8 is adapted to permit movement of both bone fragments 3, 4 toward each other, i.e. permit so called dynamic compression, which is necessary or at least essential for being able to compress the injured bone fragment portions 6, 7 such that said bone fragment portions are completely or partly reduced, while the muscular system of the patient through contractions impulsively subject the bone fragments 3, 4 to forces directed towards each other when the patient awakes from the anaesthesia. Said so called dynamic compression of the bone fragments 3, 4, which might occur also when the patient leans on the injured leg, is essential for a quick and safe healing procedure.

In order to make the abovementioned functions possible, the illustrated implant 8 comprises primarily an elongated member 9 (usually called plate) and a sleeve like member 10 protruding obliquely from said elongated member 9. The elongated member 9 is adapted to be secured by screws to the bone fragment 3 on one side of the femur fracture 2 by means of e.g. four screws (usually called cortical screws) and the sleeve like member 10 is adapted for location at the bone fragment 4 on the other side of the femur fracture 2 by means of a bone screw 12 (usually called lag screw) having an unthreaded portion 12a and a threaded portion 12b. The threaded portion 12b of the bone screw 12 is adapted to be screwed into and secured to the femoral head 5 and the unthreaded portion 12a is adapted to engage a hole 13 in the sleeve like member 10. Said hole 13 and the unthreaded portion 12a are preferably designed such that the sleeve like member 10 and the bone screw 12 can be displaced linearly or substantially linearly, but not turn or rotate relative to each other. This design and function is already known and therefore, not further described.

The elongated member 9 has an inner side which is adapted to engage the bone fragment 3, and an outer side 15 which is parallel or substantially parallel with the inner side 14.

The elongated member 9 of the implant 8 is provided with a suitable number, e.g. four, elongated holes 16 of which each hole is adapted for receiving a screw 1. The holes 16 are elongated in the longitudinal direction of the elongated member 9 and they have concave or substantially concave contact surfaces 17. The screws 1 have heads 18 with convex contact surfaces 19 which are provided with an inner edge 19a. The shape of the contact surfaces 17 of the elongated member 9 and of the contact surfaces 19 of the screw heads 18 are adapted to each other. The contact surfaces 17 of the elongated member 9 have an inner edge 20 through which they border on inner portions 21 of the elongated holes 16.

Each contact surface 19 and/or its inner edge 19a is provided inclined relative to the inner side 14 of the elongated member 9 such that the distance A between the contact surface 17 and/or its inner edge 20 at the rear portions 16a of the elongated hole 16 and the inner side 14 is greater than the distance B between the contact surface 17 and/or its inner edge 20 and said inner side 14 at the front portions 16b of the elongated hole 16. Due to this inclination of the contact surface 19 and/or its inner edge 19a, the following is achieved: when the screws 11 have been located in the rear portions 16a of the elongated holes 16, the screws are tightened with the required tightening force such that the implant 8 is sufficiently firmly fixed at the bone fragment 3 of the femoral shaft 1. Some time after this operation, the bone fragment 3 should be able to move in a forward direction F relative to the elongated member 9 and this is made possible while the screws 11 can move in forward direction F in the elongated holes 16. However, the contact surfaces 17 of the elongated member 9 might counteract this since the screws 11 have been tightened for fixation of the implant 8 at the femur fracture 2. This is due to the fact that the contact surfaces 19 of the screws 11 will engage the contact surfaces 17 of the elongated member 9 during the movement of the screws 11 in the elongated holes 16 and while there is friction between said contact surfaces 17, 19.

Thanks to the abovementioned inclination of the contact surfaces 17 of the elongated member 9 and/or their inner edges 20, the heads 18 of the screws 11 can move freely relative to said contact surfaces of the elongated member 9 or at least with a decreasing pressure there against when the screws 11 leave the rear portions 16a in the elongated holes 16 and move in forward direction F. This is illustrated in FIG. 2, where the screw 11 is shown in a rear position D in the rear portions 16a and with dashed and dotted lines in a position E somewhat further on. In position D, the contact surfaces 19 of the screw 11 and their inner edges 19a engage the contact surfaces 17 of the elongated member 9. When the screw 11 moves from position D to position E, the screw head 18 will move in parallel or substantially in parallel with the inner side 14 of the elongated member 9, which means that the inclination of the contact surfaces 17 and their inner edges 20 results in that the contact surface 19 of the screw head 18 and the edge 19a of said contact surface 19 will lie above the contact surfaces 17 and their inner edges 20. Hereby, the screws 11 can move in said forward direction F without this movement being counteracted or substantially counteracted by the friction between the contact surfaces 19 of the screw heads 18 and the contact surfaces 17 of the elongated member 9.

The angle δ of inclination between the contact surfaces of the elongated member 9 and/or the inner edges 20 of said contact surfaces, and the inner side 14 of the elongated member 9, is preferably between 1° and 6°.

The implant 8 may be designed such that the angle α between a longitudinal centre line CL of the sleeve like member 10 and the inner side 14 of the elongated member 9 is 95°±4° (in FIG. 1, the angle α between the centre line CL and a line P parallel with the inner side 14 is illustrated).

The hole 13 of the sleeve like member 10 and the unthreaded portion 12a of the bone screw-12 may be designed such that said unthreaded portion 12a engage the hole 13 displaceably but not rotatably.

The invention is not limited to the embodiment of the implant 8 described above and illustrated in the drawings, but may vary within the scope of the subsequent claims. It should be mentioned that the implant 8 may be of another type and be adapted for other femur fractures than subtrochantary, that the shape of the contact surfaces 17, 19 of the elongated member 9 and the screws 11 may be another than the one described and that the elongated member 9 may have a hole 22 closest to the sleeve like member 10 for a screw which is not shown in this embodiment.

1. Implant for fixation of bone fragments at femur fractures, preferably subtrochantary femur fractures,
wherein the implant (8) comprises an elongated member (9) and integral therewith, a sleeve like member (10),
wherein the sleeve like member (10) is adapted for location at a bone fragment (4) including the head (5) of femur and wherein the elongated member (9) is adapted for location at a bone fragment (3) including the femoral shaft (1),
wherein the implant (8) comprises a bone screw (12) having a threaded portion (12b) which is adapted to be screwed into and secured to the femoral head (5),
wherein the sleeve like member (10) has a hole (13) which is adapted for receiving an unthreaded portion (12a) of the bone screw (12),
wherein the elongated member (9) has an inner side (14) and an outer side (15), said inner side (14) being adapted to engage the bone fragment (3) including the femoral shaft (1),
wherein the elongated member (9) of the implant (8) has elongated holes (16) for screws (11) by means of which said elongated member (9) can be tightened to the bone fragment (3) with the femoral shaft (1),
wherein the screws (11) have heads (18) with contact surfaces (19) which are adapted to engage contact surfaces (17) on the elongated member (9),
wherein the screws (11) are adapted for initial location in such rear portions (16a) of the elongated holes (16) which are situated farthest away from the sleeve like member (10),
wherein the screws (11) are adapted for tightening when they are located in said rear portions (16a) of the elongated holes (16) such that the contact surfaces (19) on their heads (18) engage the contact surfaces (17) the elongated member (9) for fixation of said elongated member (9) at the bone fragment (3) with the femoral shaft (1), and
wherein the elongated holes (16) are provided such that the screws (11) can be moved therein in a forward direction (F) towards the sleeve like member (10) of the implant (8) during displacement of the bone fragment (3) with the femoral shaft (1) in said forward direction (F) relative to the elongated member (9),
characterized in
that each contact surface (17) on the elongated member (9) is, inclined relative to the inner side (14) of said elongated member (9) such that the distance (A) between said contact surface (17) at the rear portions (16a) of the elongated hole (16) and said inner side (14) is greater than the distance (B) between said contact surface (177) and said inner side (14) at front portions (16b) of the elongated hole (16), such that each screw (11) can move in forward direction (F) in said elongated hole (16), without said movement being counteracted or substantially counteracted by the contact surface (17) of the elongated member (9).

2. Implant according to claim 1, wherein the contact surfaces (19) of the screw heads (18) are convex and the contact surfaces (17) of the elongated member (9) concave, and wherein the shape of the convex and concave contact surfaces (19, 17) respectively, of the screw heads (18) and the elongated member (9) are adapted to each other,
characterized in
that an inner edge (20) of the contact surfaces (17) of each elongated hole (16), through which inner edge (20) said contact surfaces (17) of the elongated member (9) border on inner portions (21) of the elongated hole (16), is inclined relative to the inner side (14) of the elongated member (9) such that the distance (A) between said inner edge (20) at the rear portions (16a) of the elongated hole (16) is greater than the distance (B) between said inner edge (20) and said inner side (14) at the front portions (16b) of the elongated hole (16).

3. Implant according to claim 1 and/or 2, characterized in that the contact surfaces (17) of the elongated member (9) and/or an inner edge (20) of said contact surfaces (17), through which said contact surfaces (17) border on inner portions (21) of the elongated hole (16), has an angle (8) of inclination relative to the inner side (14) of the elongated member (9) of between 1° and 6°.

4. Implant according to any preceding claim, characterized in that the sleeve like member (10) thereof is provided relative to its elongated member (9) such that a longitudinal centre line (CL) of the sleeve like member (10) defines an angle (α) of 95°±4° with the inner side (14) of the elongated member (9).

5. Implant according to any preceding claim, characterized in that the sleeve like member (10) has a hole (13) and the bone screw (12) an unthreaded portion (12a) which is located in said hole (13) such that the bone screw (12) can move in axial direction relative to the sleeve like member (10).

6. Implant according to claim 5, characterized in that the unthreaded portion (12a) of the bone screw (12) and the sleeve like member (10) are designed such that said unthreaded portion (12a) of the bone screw (12) non-rotatably engages the hole (13) of the sleeve like member (10).
